(19) United States
(12) Patent Application Publication
(10) Pub. No.: US 2023/0255685 A1
(43) Pub. Date: Aug. 17, 2023

(54) ELECTROSURGICAL DEVICE WITH AUTOMATIC SHUT-OFF

(71) Applicant: Boston Scientific Medical Device Limited, Galway (IE)

(72) Inventors: Daniel Wing Fai Mok, Mississauga (CA); Gareth Davies, Toronto (CA); John Paul Urbanski, Toronto (CA); Eduardo Moriyama, Richmond (CA); Patrick Ryan, Toronto (CA); Matthew DiCicco, Guelph (CA)

(21) Appl. No.: 18/300,972

(22) Filed: Apr. 14, 2023

Related U.S. Application Data
(63) Continuation of application No. PCT/IB2021/059484, filed on Oct. 15, 2021.
(60) Provisional application No. 63/091,997, filed on Oct. 15, 2020.

(51) Int. Cl. A61B 18/14

(52) U.S. Cl. CPC A61B 18/1492; A61B 2018/144; A61B 2018/00773

(57) ABSTRACT

A puncturing device configured to create a puncture in a tissue comprising an elongate member comprising a proximal portion defining a longitudinal axis along the length of the elongate member. The elongate member further comprises a flexible distal portion of the that curves away from the longitudinal axis and a distal tip configured to deliver energy to the tissue. A sensing element placed on the flexible distal portion of the elongate member detects the curvature of the distal portion such that when the flexible distal portion of the elongate member is straightened, energy is delivered to the distal tip and when the flexible distal portion of the elongate member is curved, energy is not delivered to the distal tip.

### CROSS-REFERENCE TO RELATED APPLICATIONS

This application is a continuation of and claims the benefit of International Application Number PCT/IB2021/059484, entitled “AN ELECTROSURGICAL DEVICE WITH AUTOMATIC SHUT-OFF,” and filed Oct. 15, 2021, which claims the benefit of U.S. Provisional Application No. 63/091,997, entitled “AN ELECTROSURGICAL DEVICE WITH AUTOMATIC SHUT-OFF,” and filed Oct. 15, 2020, which are hereby incorporated by reference in their entireties.

### TECHNICAL FIELD

The disclosure relates to a surgical perforation device, configured to deliver energy to a living tissue wherein the delivery of energy is controlled by the curvature of the distal portion of the device. More specifically, the invention relates to a device and method for creating a perforation in the atrial septum while using the curvature of the distal portion of the device to automatically stop the delivery of energy to the atrial septum upon completion of the puncture.

### BACKGROUND OF THE ART

Certain medical procedures require the use of a medical device that can create punctures or channels through tissues of the heart. Specifically, puncturing the septum of a heart creates a path to the left atrium where a variety of cardiology procedures take place. One device that assists in gaining access to the left atrium is a radiofrequency (RF) transseptal puncturing device. In such devices RF energy from a generator is delivered to a target tissue to create the perforation. In operation, the user positions the puncturing device at a target location on the fossa ovalis located on the septum of the heart and turns on the generator to begin delivering energy to the target location. The delivery of RF energy to a tissue results in vaporization of the intracellular fluid of the cells which are in contact with the device. Ultimately, this results in a void, hole, or channel at the target tissue site.

Currently, the parameters around the delivery of energy involve 1) the duration of the energy delivery, and 2) pulsed or constant delivery of energy. Typically, the user will select the parameters, for example constant energy delivery for the duration of two seconds, prior to performing the puncture. The user activates the delivery via a push of a button on the generator or via a foot pedal. When the duration of energy delivery has been completed, the user will check, using various means (e.g., fluoroscopy, pressure readings, ultrasound, or contrast injections) to determine if the puncture was successful. If it was unsuccessful, the user will manually activate the energy delivery again. Once the duration is completed, the user will once again check to see if the puncture was successful. The user has the ability to turn off the delivery of energy before the duration is complete, using the button on the generator or the foot pedal, but there is still no way to confirm during the delivery of energy if the puncture was successful or not. This lack of knowledge around the success of the puncture during energy delivery may lead to inadvertent damage to surrounding tissues. For example, if the duration has been set for two seconds but the puncture has been completed in one second, the puncturing device is still delivering energy for additional time after entering the left atrium which may lead to inadvertent perforation of within the left atrium. Inadvertent perforation of other tissues of the heart may result in general tissue damage within the left atrium, ancillary device damage (i.e., damage to pacemaker leads located in atrium) or potentially critical complications such as cardiac tamponade or inadvertent aortic perforation. A cardiac tamponade is a life-threatening complication of transseptal punctures which occurs when a perforation is created at the left atrial wall, left atrial roof, or left atrial appendage. This perforation of the atrial wall may lead to an accumulation of fluid within the pericardial cavity around your heart. This buildup of fluid compresses your heart which in turn reduces the amount of blood able to enter your heart. An inadvertent aortic perforation is a rare life-threatening complication where the puncturing device enters and perforates the aorta which may require surgical repair.

In light of these potential complications associated with inadvertent damage to surrounding tissues, there exists a need to provide a novel radiofrequency puncturing device wherein the delivery of radiofrequency energy is deactivated automatically after the puncture device has completed the puncture and entered the left atrium

### BRIEF DESCRIPTION OF THE DRAWINGS

In order that the invention may be readily understood, embodiments of the invention are illustrated by way of examples in the accompanying figures, in which:

FIG. 1 is an illustration of a system used when creating a transseptal puncture to gain access to the left atrium of a patient.

FIG. 2a is an illustration of the construction of a puncturing device with a strain gauge.

FIG. 2b is an illustration of a J-tip guidewire with a strain gauge.

FIG. 2c is an illustration of a pig-tail guidewire with a strain gauge.

FIG. 3a is an illustration of a J-tip guidewire with a strain gauge affixed to the core wire, under the insulation.

FIG. 3b is an illustration of a J-tip guidewire with a strain gauge affixed to the exterior of the insulation.

FIG. 4a is an illustration of a puncturing device constrained by the sheath and dilator.

FIG. 4b is an illustration of a puncturing device unconstrained by the sheath and dilator.

FIG. 5 is an illustration of an example computer algorithm to control the shut off of energy delivery.

FIG. 6a is an illustration of a cross sectional view of a puncturing device wherein the distal portion comprises a conductive wire surrounded by a conductive coil.

FIG. 6b is an illustration of a cross-sectional view of a puncturing device where the distal portion has been constrained, resulting in contact between the conductive coil and conductive wire.

### DETAILED DESCRIPTION

Various minimally invasive procedures involve creating a perforation in a living tissue. One such procedure is performing a transseptal puncture which allows surgeons to gain access to the left side of the heart by creating a puncture from the right side of the heart through the septum. Recently, medical devices have been configured to perform the puncture by delivering energy, specifically radiofrequency energy, to the tissue. The delivery of radiofrequency energy to a tissue results in vaporization of the intracellular fluid of the cells which are in contact with the energy delivery device. This results in a perforation at the target tissue site. One of the complications which may arise during a transseptal puncture is the inadvertent puncturing of the left atrial wall or aorta. These potentially life-threatening complications may result in damage to surrounding tissue or ancillary devices, or perforation of the left atrial wall or aorta.

The problem of inadvertent puncturing of the left atrium is solved by providing an electrosurgical puncturing device with a mechanism to shut off the delivery of energy after the puncture of the septum has been completed.

In one broad aspect, embodiments of the present invention comprise a puncturing device configured to create a puncture in a tissue. The puncturing device has an elongate member comprising a proximal portion defining a longitudinal axis along the length of the elongate member. The elongate member further comprises a flexible distal portion that curves away from the longitudinal axis and a distal tip configured to deliver energy to the tissue. A sensing element is placed on the flexible distal portion of the elongate member such that the sensing element detects the curvature of the distal portion. When the flexible distal portion is straightened, the energy is delivered to the distal tip and when the flexible distal portion is curved, energy is not delivered to the distal tip.

As a feature of this broad aspect the sensing element is a strain gauge.

As another feature of this broad aspect, the elongate member is composed of a conductive material. In some embodiments, the elongate member comprises a layer of insulation overtop the conductive material. In some embodiments, the sensing element is positioned overtop of the layer of insulation. In an alternative embodiment, the sensing element is positioned underneath the layer of insulation.

As a feature of this aspect, the sensing element is positioned on a side of the flexible distal portion that undergoes compression when curved. In an alternative embodiment, the sensing element is positioned on the side of the flexible distal portion that undergoes tension when curved.

As another feature of this broad aspect, the puncturing device is a guidewire. In some embodiments, the guidewire is a J-tip guidewire. In alternative embodiments, the guidewire is a pig-tail guidewire.

In another broad aspect, embodiments of the present invention comprise a puncturing device configured to create a puncturing in a tissue comprising an elongate member, composed of a conductive core wire. The elongate member comprises a proximal portion defining a longitudinal axis along the length of the elongate member. The elongate member further comprises a flexible distal portion that curves away from the longitudinal axis. The flexible distal portion comprises a conductive coil that surrounds the conductive core wire. The flexible distal portion ends in a distal tip configured to deliver energy to the tissue, wherein when the flexible distal portion of the elongate member is straightened, the conductive coil contacts the conductive core wire, enabling energy deliver to the distal tip. When the flexible distal portion of the elongate member is curved, the conductive coil does not contact the conductive core wire, disabling energy delivery to the distal tip.

As another broad aspect, embodiments of the present invention comprise a puncturing assembly for puncturing a tissue. The puncturing assembly comprises a puncturing device. The puncturing device comprises an elongate member having a proximal portion defining a longitudinal axis along the length of the elongate member. The puncturing device further comprises a flexible distal portion and a sensing element placed on the flexible distal portion such that the sensing element detects curvature of the flexible distal portion. The flexible distal portion ends in a distal tip, configured to deliver energy to the tissue. The puncturing assembly further comprises a supporting member comprising a lumen configured to receive the puncturing device such that the flexible distal portion of the puncturing device is constrained to a straightened configuration when received within the lumen of the supporting member.

As a feature of this broad aspect, the flexible distal portion is constrained within the supporting member, energy is enabled, and when the flexible distal portion is unconstrained, energy delivery is disabled.

As another feature of this broad aspect, the supporting member comprises a dilator.

As a feature of this broad aspect, the puncture device comprises a puncturing guidewire. In some embodiments, the puncturing guidewire comprises a J-tip guidewire. In an alternative embodiment, the puncturing guidewire comprises a pig-tail guidewire.

As another feature of this broad aspect, the sensing element is a strain gauge.

As another feature of this broad aspect, the elongate member is composed of a conductive material. In some embodiments, the elongate member comprises a layer of insulation overtop the conductive material. In some embodiments, the sensing element is positioned overtop of the layer of insulation. In an alternative embodiment, the sensing element is positioned underneath the layer of insulation.

As a feature of this aspect, the sensing element is positioned on a side of the flexible distal portion that undergoes compression when curved. In an alternative embodiment, the sensing element is positioned on the side of the flexible distal portion that undergoes tension when curved.

In another broad aspect, embodiments of the present invention comprise a method for puncturing a septum of a heart using a puncturing assembly comprising a puncture device contained within a lumen of a supporting member. The method comprises the steps of: (i) gaining access to a vasculature of a patient; (ii) advancing the puncturing assembly to a target location on the septum, such that a distal tip of the puncturing device, configured to deliver energy, is exposed outside a distal tip of the supporting member while a flexible, curved, distal portion of the puncturing device remains constrained within the supporting member lumen; wherein the flexible, curved, distal portion of the puncturing device comprises a sensing element to detect the curvature of the distal portion; (iii) delivering energy to the distal tip of the puncturing device such that a puncture is created at the target location; and, (iv) advancing the puncturing device such that the flexible, curved, distal portion of the puncturing device is no longer constrained within the lumen of the supporting member. The sensing element detects the unconstrained curvature of the flexible, curved, distal portion of the puncturing device and disables the delivery of energy to the distal tip of the puncturing device.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

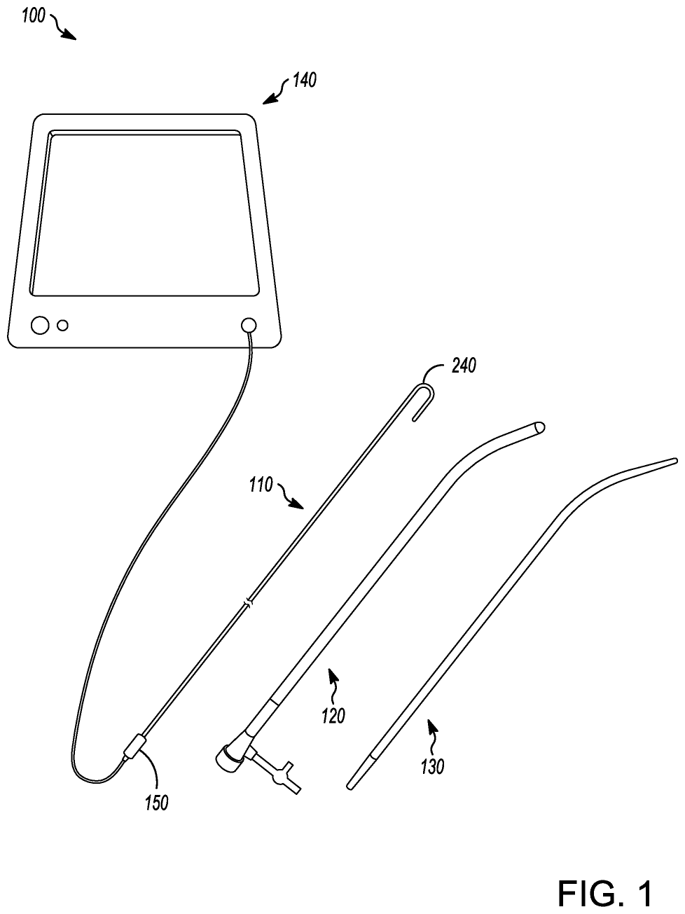

FIG. 1 illustrates an embodiment of an exemplary system 100 that may be used to access the left atrium via transseptal puncture. The system 100 comprises a puncturing device 110, a sheath 120, a dilator 130, and an energy generator 140 which is connected to the puncturing device through a connection means 150. The puncturing device 110, for example a pig-tail guidewire (not shown) or J-tip guidewire, is configured for delivering energy to a tissue (such as the atrial septum of a patient's heart). Energy is delivered from the generator 140 to an energy delivery device located at the distal tip of the puncturing device 110. In this embodiment, the puncturing device 110 comprises a sensing element located at the distal portion 240. The sensing element is configured to detect the curvature of the distal portion 240 on which it is attached and sends a signal to the generator 140. The signal from the sensing element varies with the curvature of the distal portion. When the distal portion is in an unconstrained state (i.e. curved distal portion 240), the generator 140 is configured to process the signal and take a corresponding action. For example, in some embodiments, when the generator 140 receives a signal corresponding to the distal portion 240 being in an unconstrained state the generator will automatically shut off the energy delivery.

An exemplary method of accessing the left atrium of a patient using the present invention may include the following steps:
(i) Gaining access to the vasculature, for example through the groin to the femoral vein.
(ii) Advancing the puncturing device 110 and assembly (i.e., sheath 120 and dilator 130) to the target location, which in an embodiment is the fossa ovalis of a patient's heart. At this stage, the distal portion 240 of the puncturing device 110 is straightened, constrained by the dilator 130 and sheath 120 assembly. The distal portion 240 comprises a pre-determined non-linear shape when it is not constrained.
(iii) Energy is delivered from the generator 140, through the puncturing device 110, and to the fossa ovalis to create a puncture in the septum.
(iv) The puncturing device 110 is advanced through the puncture, entering the left atrium; upon leaving the assembly, the distal portion 240 being no longer constrained by the sheath 120 and dilator 130, reverts back to its pre-determined non-linear shape. The sensing element detects this change in geometry and signals the generator 140 to shut off the delivery of energy.

In an alternative method, access to the heart may be gained through the superior vena cava, where the puncturing device 110 enters the vasculature via the subclavian vein. Persons skilled in the art will appreciate that the dimensions of the assembly and puncture device may be varied depending on where the vasculature is accessed (e.g., subclavian vein), and the anatomy (e.g., the right atrium of the heart).

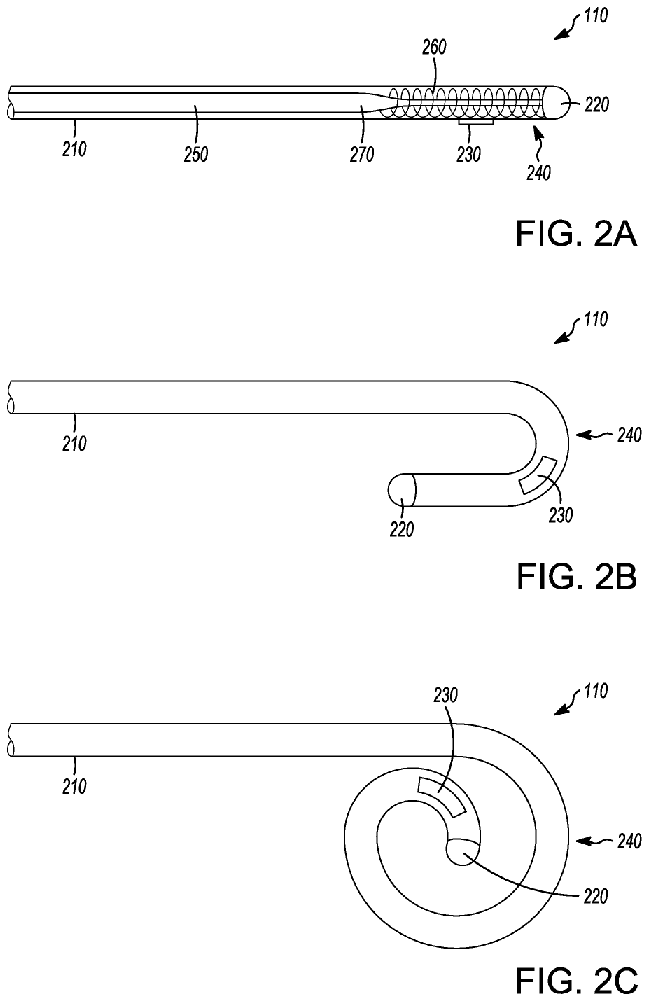

Various embodiments of the invention used in the system 100 and method described above can be seen in FIG. 2a-2c. With reference to FIG. 2a, the puncturing device 110 is comprised of an elongate member 250, such as a wire, coated in electrically insulating material 210 which substantially covers the conductive elongate member, exposing a portion of the distal tip to form an electrode 220. The elongate member 250 may further comprise a taper 270, providing flexibility, in the distal portion 240. In addition to the taper 270, the distal portion 240 of the elongate member 250 may comprise a coil 260 to provide support. The elongate member 250 and coil 270 may both be comprised of an electrically conductive material, such as nitinol or stainless steel, to allow for energy to be delivered from the generator, along the elongate member 250, to the electrode 220. The coating 210 is comprised of an electrically insulating material, such as PTFE (polytetrafluoroethylene) coating, to ensure that the delivery of radiofrequency energy travels along the length of the puncturing device 110 to the exposed electrode tip 220. Alternatively, the elongate member 250 may not have an insulative coating applied to it; rather the sheath or dilator may be comprised of a non-electrically conductive material to ensure that energy is delivered through the distal tip of the puncturing device 110. In an alternative embodiment, the elongate member 250 may be comprised of a non-electrically conductive material, such as polyetheretherketone (PEEK) or polyimide. In this alternative embodiment, there would need to be a conductive element to deliver the energy to the distal tip (e.g., an insulated wire). The distal region 240 may be formed during manufacturing, typically exposing it to heat while it is fixed in a desired shape, such that there is a curve which curls away from a central axis. The electrode 220 may be coupled to a conductive wire which carries the energy from the generator to the electrode 220 at the distal tip of the puncturing device 110. A sensing element 230 is attached to the distal portion 240 of the puncturing device 110, such that it is exposed to the change in geometry as the puncturing device 110 moves from a constrained state (i.e., inside the sheath and/or dilator, straightened as illustrated in FIG. 2a) to an unconstrained state (e.g., curved state as illustrated in FIG. 2b or 2c). The sensing element 230 may comprise a strain gauge; those skilled in the art will appreciate that other sensing means may be used to detect the change in geometry of the distal portion 240.

With reference now to FIG. 2b, the puncturing device 110 has a distal portion 240 that has been shaped in a J-tip configuration when unconstrained. The sensing element 230 is attached such that when the distal portion 240 is in its unconstrained configuration (e.g., a J.-tip configuration), the sensing element 230 bends or distorts with the curve.

The puncturing device 110 may comprise a distal portion 240 that has a pig-tail configuration when unconstrained, as illustrated in FIG. 2c. The sensing element 230 is preferably placed at the distal most curved section along the distal portion 240, such that the sensing element 230 bends immediately as the puncturing device 110 begins to curl. The change in shape of the sensing element 230 is detected as the distal portion 240 becomes unconstrained. In response to detecting the change in shape, the generator can operate in “auto-off” mode and stop delivering RF energy automatically. This configuration allows the energy delivery to be shut off as soon as the puncturing device 110 enters the left atrium, reducing the likelihood that the puncturing device 110 damages surrounding tissues by inadvertently delivering RF.

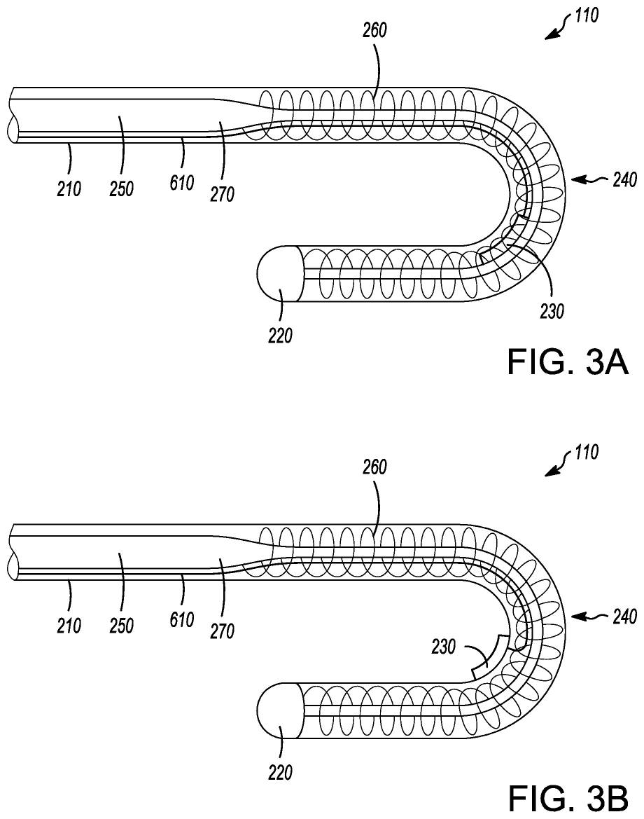

In some embodiments, the sensing element 230 may be affixed to the elongate member 250 directly, as illustrated in FIG. 3a. For example, the sensing element 230 may be welded or glued to the elongate member 250. Those skilled in the art will appreciate that other means may be used to affix the sensing element 230 to the elongate member 250. The insulative coating 210 may cover both the sensing element 230 as well as the insulated internal wiring 610. In an alternative embodiment, the sensing element 230 may be affixed directly to the insulative coating 210, illustrated in FIG. 3b. For example, the sensing element 230 may be affixed through welding or gluing to the insulative coating 210. The insulated internal wiring 610 may run along the length of the puncturing device 110. In an alternative embodiment, the insulated internal wiring 610 may be attached to and run along the outside of the insulative coating 210 (not shown). The insulated internal wiring 610 exits the puncturing device 110 at its proximal end, which in turn connects to the generator. The sensing element 230 is capable of detecting a change in geometry of the distal portion 240 of the puncturing device 110. In some embodiments, the sensing element 230 may be placed in the distal portion 240, on the inner or outer portion of the curvature, such that the sensing element 230 bends or distorts with the curve of the distal portion 240. In an embodiment, the internal insulated wiring 610 delivers a signal from the sensing element 230 to the generator.

In one embodiment, the sensing element 230 may comprise a strain gauge, attached to the inner portion of the curve, as illustrated in FIGS. 3a and 3b. In an alternative embodiment, the strain gauge may be positioned on the outer portion of the curve. One skilled in the art would appreciate that the positioning of the strain gauge may be anywhere along the curved portion such that there is a difference in strain gauge reading from the straightened versus the curved state. The strain gauge distorts with the curvature of the distal portion 240. The distortion of the strain gauge will cause its electrical resistance to change; for example, the strain gauge undergoing compression will result in a decrease in resistance while tension will result in an increase in resistance. This change in resistance is used to determine whether the distal portion is in its constrained configuration (i.e., conforming to the shape of the sheath and/or dilator) or in its unconstrained configuration (i.e., in its pre-determined shape). The detected signal from the strain gauge may be used to enable or disable the delivery of energy to the puncturing device 110. For example, the measurement could be implemented into an algorithm which compares a baseline strain to the measured strain. The baseline strain may be the unconstrained measure of strain, as in the amount of strain on the strain gauge when the distal portion 240 of the puncturing device 110 is curved or shaped; this measurement could be taken during manufacturing. The algorithm may then compare the measured strain to this baseline to determine if the distal portion 240 is straightened (i.e., constrained) or curved (i.e., unconstrained) to enable or disable the delivery of energy. For example, if the strain detected is more positive than the baseline strain (i.e., the change in resistance is positive, meaning the strain gauge is undergoing tension) would correspond to the distal portion 240 of the puncturing device 110 being straightened or constrained; thus, the delivery of energy is enabled. If the strain detected is the same as the baseline strain, it would indicate that the distal portion 240 of the puncturing device 110 is curved, or unconstrained. Upon detection that the current strain of the device is equivalent to the baseline strain, the generator may be configured to disable the delivery of energy to the puncturing device.

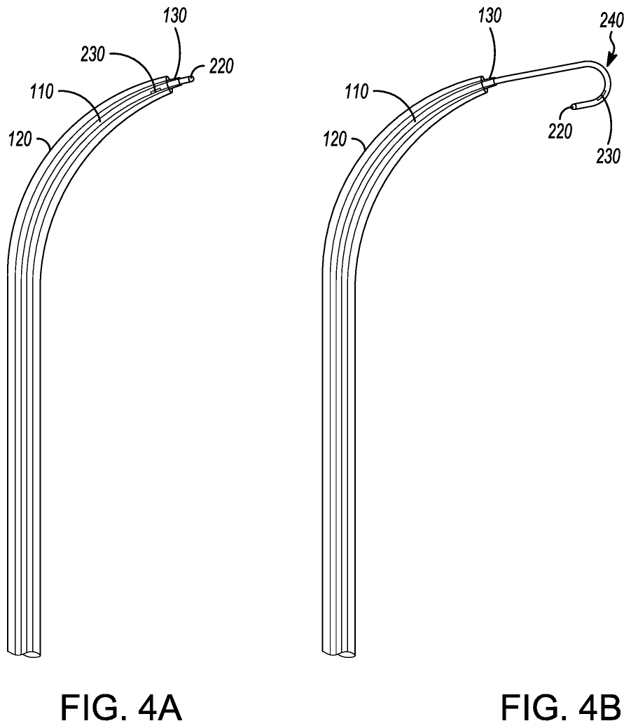

The constrained and unconstrained state of the puncturing device 110 is illustrated in FIGS. 4a and 4b, respectively. In this embodiment, the puncturing device 110 is constrained by the dilator 130 when inserted into the lumen of the ancillary device. The flexibility of the distal portion 240 of the puncturing device 110 results in the straightening of the, normally, curved distal portion 240. This causes the sensing element 230 to be straightened as well. In some embodiments, this configuration (as seen in FIG. 4a) of the puncturing device 110 is primed to perform the puncture. The configuration of the sensing element 230 indicates that the puncturing device 110 is in position to have energy delivered to the electrode 220. For example, if the sensing element 230 is a strain gauge, the strain detected would be greater than the baseline strain (i.e., the strain detected is of the puncture device in an unconstrained configuration). The generator would receive this information and enable the delivery of energy. In some embodiments, energy delivery may be initiated by the user. For example, the generator may alert the user to begin energy delivery via sound, user interface prompt, optical alert (i.e., light turning on), or any other means of alert. In an alternative embodiment, energy delivery may be automatic such that once the sensing element is in the constrained configuration, energy is delivered. Upon completion of a puncture, the puncturing device 110 is pushed through the hole in the septum and enters the left atrium. The distal portion 240 of the puncturing device 110 is pushed out of the dilator 130 and into the left atrium. As the puncturing device 110 enters the left atrium, the distal portion 240 is no longer constrained and reverts back to its original shape (FIG. 4b). The curving of the distal portion 240, bends sensing element 230; the sensing element 230 detects a change in the configuration of the distal portion. In an embodiment, the detected signal is interpreted as the puncturing device 110 having completed the puncture and the delivery of energy should be shut off. For example, if the sensing element 230 is a strain gauge, the strain detected would be approximately equal to the baseline strain (obtained when the device is in an unconstrained state). In response to detecting this state, the generator could be configured to disable the delivery of energy. Additionally, the generator may alert the user, notifying them that energy delivery has been disabled. This alert may be in the form of a sound, user interface prompt, optical alert (i.e., light turning off), or any other means of alert.

In some embodiments, the sensing element 230 may be positioned proximal the distal portion 240, along the elongate member 250. As an example, the sensing element 230 may be located along the elongate member 250 such that when the puncturing device 110 is in an optimal puncture position, the sensing element 230 is located within the curved portion of the dilator 130. In this configuration, the sensing element 230 detects the change from a straight configuration (i.e., when the sensing element 230 is proximal the curve of the dilator) to a curved configuration (i.e., when the sensing element 230 is contained within the curve of the dilator). In this embodiment, when the sensing element 230 is in the straight configuration, no energy is delivered to the electrode 220. When the sensing element 230 is in a curved configuration, energy may be delivered; in other words, when the sensing element 230 is positioned within the curved portion of the dilator 130 (when the puncturing device 110 is in the optimal position for puncturing tissue) energy may be delivered to the electrode 220, enabling the device 110 to perform a puncture. Once the puncture is completed, the puncturing device 110 may be advanced and the sensing element 230 moves from a curved configuration (e.g., positioned within the curved portion of the dilator 130) to a straight configuration (e.g., positioned to within the straight portion of the dilator 130 that is distal the curved portion) which, in turn, disables the delivery of energy. In an alternative embodiment, the sensing element 230 may be configured to enable the delivery of energy while in the straight configuration. As an example of this embodiment, the sensing element 230 may be positioned on the elongate member 250 such that when the puncturing device 110 is in a position optimal for puncturing tissue, the sensing element 230 is proximal to the curved portion of the ancillary device (e.g., dilator 130) and in a straight configuration, primed for delivering energy to the tissue. Upon completion of the puncture, the puncturing device 110 is advanced through the dilator 130 and enters the curved portion of the dilator 130. In a curved configuration, the sensing element 230 is configured to disable the delivery of energy. In other words, when the sensing element 230 reaches the curved portion of the dilator 130, energy delivery is disabled. In some embodiments the sensing element 230 may be positioned on top of the insulating layer 210 of the puncture device 110. In another embodiment, the sensing element 230 may be positioned beneath the insulating layer 210 of the puncture device 110. In some embodiments, the sensing element 230 may be positioned on an inner portion of the puncturing device; in other words, the sensing element 230 would undergo compression when constrained by the curved portion of the dilator 130. In an alternative embodiment, the sensing element 230 may be positioned on an outer portion of the puncturing device such that it undergoes tension when constrained by the curved portion of the dilator 130.

As previously discussed, a software algorithm may be implemented to control the delivery of energy from the generator to the puncturing device. The algorithm may use signals from the sensing element to determine the geometry of the distal portion; this in turn would be used to control the delivery of energy. For example, if the sensing element was a strain gauge placed on the curve of the distal portion, it may use strain measurements as previously described to signal the generator to enable or disable the delivery of energy.

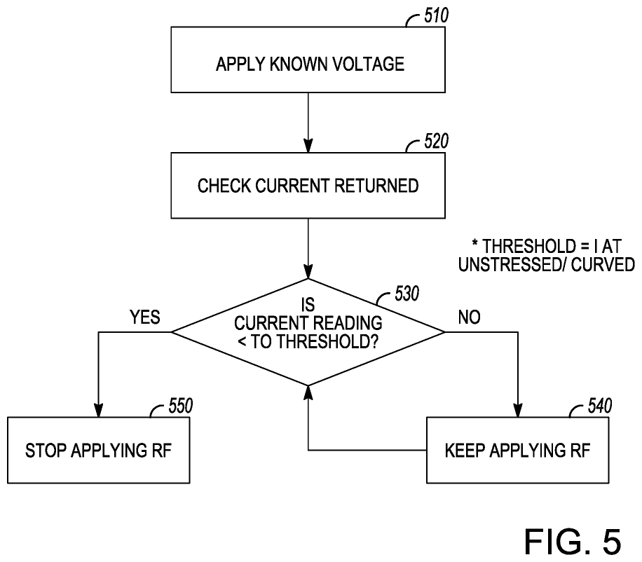

In an alternative embodiment, the generator may apply a known voltage to the strain gauge. As the strain gauge distorts, the resistance of the strain gauge would change, ultimately changing the current that is returned to the generator. A baseline of current may be determined during manufacturing and set as the value for when the puncturing device is unconstrained. This baseline would be used to shut off the delivery of energy as this value would be indicative of the puncturing device entering the left atrium after the puncture has been completed. For example, with reference now to FIG. 5, the generator would apply a known voltage to the strain gauge throughout the procedure 510. Using the known voltage and the resistance of the strain gauge, a current of the electrical signal may be calculated 520. The algorithm may compare the current of the electrical signal to see if it matches the baseline current value (i.e., unconstrained, curved distal portion) 530. As the strain gauge is put under tension, the resistance increases; therefore, the electrical current will decrease when the distal portion of the puncturing device is constrained compared to when it is unconstrained. Thus, if the measured current is less than the baseline current value, the delivery of energy is enabled 540 and the measured current continues to be compared 530. If the measured current matches the baseline current, the delivery of energy is disabled 550, signaling that the puncture has been completed and the puncturing device has entered the left atrium. Those skilled in the art will appreciate other electrical signal properties may be used and implemented in an algorithm to control the delivery of energy.

Alternatively, the delivery of energy may be implemented through hardware means. In one embodiment, the sensing element may control switches in the generator which will control the delivery of energy to the puncturing device. In some embodiments, the sensing element may comprise a strain gauge which could have a current gated switch to control the delivery of energy. The current gated switch may toggle on or off dependent on the inflexion of the strain gauge. For example, as the strain gauge is bent (i.e., the puncturing device is unconstrained), the current gated switch may toggle to shut off the delivery of energy.

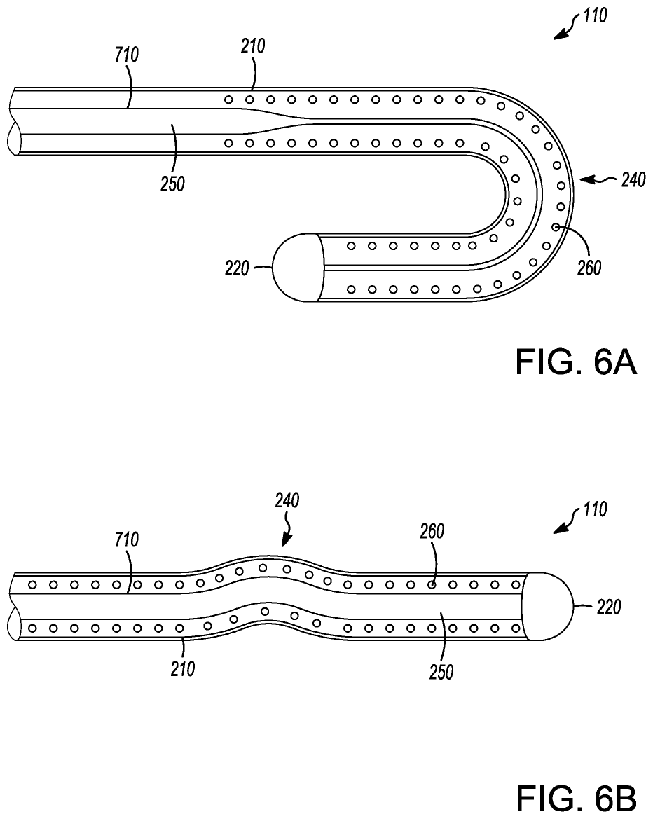

As previously described, the puncturing device 110 comprises an electrode 220 at the distal tip which may be used to deliver energy in order to puncture tissue. The puncturing device 110 further comprises an elongate member 250 which tapers 270 at the distal portion 240 (as illustrated in FIGS. 6a and 6b). A coil 260 is used to provide support to the distal portion 240. In an alternative embodiment of the present invention, the coil 260 and elongate member 250 may be composed of conductive material and a layer of insulation 210 is applied to the device. In an unconstrained state, as illustrated in FIG. 6a, the coil 260 and elongate member 250 are not contacting one another. However, in a constrained state, as illustrated in FIG. 6b, the elongate member 250 may have the tendency to kink, thereby resulting in contact between the coil 260 and the elongate member 250. In some embodiments, the coil 260 may be connected to the generator such that when the puncturing device 110 is constrained (i.e., the elongate member 250 is kinked), the contact between the coil 260 and the elongate member 250 results in energy being delivered; thus, energy would be delivered from the generator to the coil 260 which in turn would be delivered to the elongate member 250 and, ultimately, to the electrode 220 at the distal tip. Upon completion of the puncture, the puncturing device 110 would be pushed through, resulting in the unconstrained state (FIG. 6a), in which case the elongate member 250 and coil 260 are no longer in contact; thereby, halting energy delivery to the electrode 220.

In an alternative embodiment of the present invention, the puncturing device 110 comprises an electrode 220 at the distal tip, configured to deliver energy to puncture a tissue. In some embodiments the energy may delivered to the electrode 220 via a conductive wire. In some embodiments the conductive wire may be an insulated wire 610. The insulated wire 610 may be positioned on the puncture device 110 such that it runs along the outer side of the curved distal portion 240; that is, when the puncturing device 110 is unconstrained, the insulation wire 610 would be in tension. The insulated wire 610 may be comprised of two separate portions: a distal portion and a proximal portion. The insulated wire 610 may be positioned along the puncture device 110 such that when the curved distal portion 240 is constrained by an ancillary device (e.g., dilator 130) the two separate portions of the insulated wire 610 contact one another, allowing for the delivery of energy. In other words, when the puncture device 110 is in a positioned that is primed or optimal for puncture, the curved distal portion 240 of the puncture device is straightened. The straightening of the curved distal portion 240 results in the distal portion and the proximal portion of the insulated wire 610 are compressed together, thereby enabling energy delivery. Upon completion of the puncture, the puncturing device 110 is advanced out of the dilator 130 such that the curved distal portion 240 is no longer constrained and resumes its curved configuration. As a result, the distal portion and the proximal portion of the insulated wire 610 are pulled apart from one another due to the distal on the outer circumference of the curved distal portion 240 being elongated. Thus, there is a break in the circuit and energy delivery is disabled.

### FURTHER EXAMPLES

1) A puncturing device configured to create a puncture in a tissue comprising:
   An elongate member comprising a proximal portion defining a longitudinal axis along the length of the elongate member;
   A flexible distal portion of the elongate member that curves away from the longitudinal axis;
   A distal tip configured to deliver energy to the tissue; and,
   A sensing element placed on the flexible distal portion of the elongate member such that the sensing element detects curvature of the distal portion;
   Wherein when the flexible distal portion of the elongate member is straightened, energy is delivered to the distal tip and when the flexible distal portion of the elongate member is curved, energy is not delivered to the distal tip.
2) The puncturing device of example 1, wherein the sensing element is a strain gauge.
3) The puncturing device of example 1, wherein the elongate member is composed of a conductive material.
4) The puncturing device of example 3, wherein the elongate member comprises a layer of insulation overtop of the conductive material.
5) The puncturing device of example 4, wherein the sensing element is positioned overtop the layer of insulation.
6) The puncturing device of example 4, wherein the sensing element is positioned underneath the layer of insulation.
7) The puncturing device of example 1, wherein the sensing element is positioned on an inner portion of the flexible distal portion that undergoes compression when curved.
8) The puncturing device of example 1, wherein the sensing element is positioned on an outer portion of the flexible distal portion that undergoes tension when curved.
9) The puncturing device of example 1, wherein the puncturing device is a guidewire.
10) The puncturing device of example 9, wherein the guidewire is a J-tip guidewire.
11) The puncturing device of example 9, wherein the guidewire is a pig-tail guidewire.
12) A puncturing device configured to create a puncture in a tissue comprising:
   An elongate member, composed of a conductive core wire, comprises a proximal portion defining a longitudinal axis along the length of the elongate member;
   A flexible distal portion of the elongate member that curves away from the longitudinal axis;
   Wherein the flexible distal portion comprises a conductive coil surrounding the conductive core wire; and,
   A distal tip configured to deliver energy to the tissue;
   Wherein when the flexible distal portion of the elongate member is straightened, the conductive coil contacts the conductive core wire, enabling energy delivery to the distal tip, and when the flexible distal portion of the elongate member is curved, the conductive coil does not contact the conductive core wire, disabling energy delivery to the distal tip.
13) A puncturing assembly for puncturing a tissue, the puncturing assembly comprising:
   a puncturing device comprising an elongate member having a proximal portion defining a longitudinal axis along the length of the elongate member;
   the puncturing device further comprising a flexible distal portion of the elongate member that curves away from the longitudinal axis and a sensing element placed on the flexible distal portion of the elongate member such that the sensing element detects curvature of the flexible distal portion;
   wherein the flexible distal portion ends in a distal tip configured to deliver energy to the tissue; and,
   a supporting member comprising a lumen configured to receive the puncturing device;
   wherein the flexible distal portion is constrained to a straightened configuration when received within the lumen of the supporting member.
14) The puncturing assembly of example 13, wherein when the flexible distal portion is constrained within the supporting member, energy delivery is enabled and when the flexible distal portion is unconstrained, energy delivery is disabled.
15) The puncturing assembly of example 13, wherein the supporting member comprises a dilator.
16) The puncturing assembly of example 13, wherein the puncturing device comprises a puncturing guidewire.
17) The puncturing assembly of example 16, wherein the puncturing guidewire comprises a J-tip guidewire.
18) The puncturing assembly of example 16, wherein the puncturing guidewire comprises a pig-tail guidewire.
19) The puncturing assembly of example 13, wherein the sensing element is a strain gauge.
20) The puncturing device of example 13, wherein the elongate member is composed of a conductive material.
21) The puncturing device of example 20, wherein the elongate member comprises a layer of insulation overtop of the conductive material.
22) The puncturing device of example 21, wherein the sensing element is positioned overtop the layer of insulation.
23) The puncturing device of example 21, wherein the sensing element is positioned underneath the layer of insulation.
24) The puncturing device of example 13, wherein the sensing element is positioned on an inner portion of the flexible distal portion that undergoes compression when curved.
25) The puncturing device of example 13, wherein the sensing element is positioned on an outer portion of the flexible distal portion that undergoes tension when curved.
26) A method for puncturing a septum of a heart using a puncturing assembly comprising a puncturing device contained within a lumen of a supporting member, the method comprising the steps of:
   (v) Gaining access to a vasculature of a patient;
   (vi) Advancing the puncturing assembly to a target location on the septum such that a distal tip of the puncturing device, configured to deliver energy, is exposed outside a distal tip of the supporting member while a flexible, curved, distal portion of the puncturing device remains constrained within the supporting member lumen;
   wherein the flexible, curved, distal portion of the puncturing device comprises a sensing element to detect the curvature of the distal portion;
   (vii) Delivering energy to the distal tip of the puncturing device such that a puncture is created at the target location; and
   (viii) Advancing the puncturing device such that the flexible, curved, distal portion of the puncturing device is no longer constrained within the lumen of the supporting member;
   whereby the sensing element detects the unconstrained curvature of the flexible, curved, distal portion of the puncturing device and disables the delivery of energy to the distal tip of the puncturing device.
27) An assembly for puncturing a target tissue, the assembly comprising:
   a puncturing device, the puncturing device comprising:
      an elongate member;
      a distal tip configured to deliver energy to the target tissue;
      a sensing element positioned on the elongate member;
   a supporting member, the supporting member comprising:
      a supporting member proximal portion and a supporting member distal portion with a lumen configured to receive the puncturing device extending therebetween;
      the supporting member distal portion comprising a curved portion and a straight portion distal to the curved distal portion, wherein the straight portion;
      an open distal end;
   and, wherein when the puncturing device is inserted into the supporting member, the sensing element detects a change in curvature of the puncturing device as the puncturing device is advanced through the supporting member distal portion.
28) The assembly of example 27, wherein the sensing element is positioned on the elongate member such that when the distal tip of the puncturing device protrudes from the open distal end of the supporting member, the sensing element is located within the curved portion of the supporting member.
29) The assembly of example 28, wherein the sensing element is configured to enable energy delivery when constrained within the curved portion of the supporting member and disable energy delivery when unconstrained by the curved portion.
30) The assembly of example 27, wherein the sensing element is positioned on the elongate member such that when the distal tip of the puncturing device protrudes from the open distal end of the supporting member, the sensing element is located proximal to the curved portion of the supporting member.
31) The assembly of example 30, wherein the sensing element is configured to enable energy delivery when unconstrained by the curved portion and disable energy delivery when constrained by the curved portion.
32) The assembly of any one of examples 27 to 31, wherein the supporting member is a dilator.
33) The assembly of any one of examples 27 to 32, wherein the sensing element is positioned underneath a layer of insulation of the puncturing device.
34) The assembly of any one of examples 27 to 32, wherein the sensing element is positioned overtop a layer of insulation of the puncturing device.
35) The assembly of any one of examples 27 to 34, wherein the sensing element is positioned on an outer portion of the puncturing device such that it undergoes tension when curved by the curved portion.
36) The assembly of any one of examples 27 to 34, wherein the sensing element is positioned on an inner portion of the puncturing device such that it undergoes compression when curved by the curved portion.
37) The assembly of any one of examples 27 to 36, wherein the puncturing device is a flexible J-tip guidewire.
38) The assembly of any one of examples 27 to 36, wherein the puncturing device is a flexible pig-tail guidewire.
39) A puncturing device for puncturing a target tissue, the puncturing device comprising:
   an elongate member comprising a proximal portion defining a longitudinal axis along the length of the elongate member;
   a flexible distal portion of the elongate member that curves away from the longitudinal axis;
   a distal tip configured to deliver energy to the tissue;
   a first conductive wire extending along the proximal portion of the elongate member, wherein the first conductive wire ends at a distance along the flexible distal portion;
   a second conductive wire coupled to the distal tip, wherein the second conductive wire ends distal to the first conductive wire;
   wherein the first and second conductive wire are positioned along an outer edge of the flexible distal portion;
   whereby, when the flexible distal portion is straightened, the first conductive wire contacts the second conductive wire, thereby enabling energy delivery; and,
   whereby, when the flexible distal portion is curved, the first conductive wire does not contact the second conductive wire, thereby disabling energy delivery.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A puncturing device configured to create a puncture in a tissue comprising:
an elongate member comprising a proximal portion defining a longitudinal axis along the length of the elongate member;
a flexible distal portion of the elongate member that curves away from the longitudinal axis;
a distal tip configured to deliver energy to the tissue; and,
a sensing element placed on the flexible distal portion of the elongate member such that the sensing element detects curvature of the distal portion;
wherein when the flexible distal portion of the elongate member is straightened, energy is delivered to the distal tip and when the flexible distal portion of the elongate member is curved, energy is not delivered to the distal tip.

2. The puncturing device of claim 1, wherein the sensing element is a strain gauge.

3. The puncturing device of claim 1, wherein the elongate member is composed of a conductive material.

4. The puncturing device of claim 3, wherein the elongate member comprises a layer of insulation overtop of the conductive material.

5. The puncturing device of claim 4, wherein the sensing element is positioned overtop the layer of insulation.

6. The puncturing device of claim 4, wherein the sensing element is positioned underneath the layer of insulation.

7. The puncturing device of claim 1, wherein the sensing element is positioned on an inner portion of the flexible distal portion that undergoes compression when curved.

8. The puncturing device of claim 1, wherein the sensing element is positioned on an outer portion of the flexible distal portion that undergoes tension when curved.

9. The puncturing device of claim 1, wherein the puncturing device is a guidewire, a J-tip guidewire, or a pig-tail guidewire.

10. A puncturing assembly for puncturing a tissue, the puncturing assembly comprising:
a puncturing device comprising an elongate member having a proximal portion defining a longitudinal axis along the length of the elongate member;
the puncturing device further comprising a flexible distal portion of the elongate member that curves away from the longitudinal axis and a sensing element placed on the flexible distal portion of the elongate member such that the sensing element detects curvature of the flexible distal portion;
wherein the flexible distal portion ends in a distal tip configured to deliver energy to the tissue; and,
a supporting member comprising a lumen configured to receive the puncturing device;
wherein the flexible distal portion is constrained to a straightened configuration when received within the lumen of the supporting member.

11. The puncturing assembly of claim 10, wherein when the flexible distal portion is constrained within the supporting member, energy delivery is enabled and when the flexible distal portion is unconstrained, energy delivery is disabled.

12. The puncturing assembly of claim 10, wherein the supporting member comprises a dilator.

13. The puncturing assembly of claim 10, wherein the puncturing device comprises a puncturing guidewire, a J-tip guidewire, or a pig-tail guidewire.

14. The puncturing assembly of claim 10, wherein the sensing element is a strain gauge.

15. The puncturing device of claim 10, wherein the elongate member is composed of a conductive material.

16. The puncturing device of claim 15, wherein the elongate member comprises a layer of insulation overtop of the conductive material.

17. The puncturing device of claim 16, wherein the sensing element is positioned overtop the layer of insulation.

18. The puncturing device of claim 16, wherein the sensing element is positioned underneath the layer of insulation.

19. The puncturing device of claim 10, wherein the sensing element is positioned on an inner portion of the flexible distal portion that undergoes compression when curved or on an outer portion of the flexible distal portion that undergoes tension when curved.

20. A puncturing device for puncturing a target tissue, the puncturing device comprising:
an elongate member comprising a proximal portion defining a longitudinal axis along the length of the elongate member;
a flexible distal portion of the elongate member that curves away from the longitudinal axis;
a distal tip configured to deliver energy to the tissue;
a first conductive wire extending along the proximal portion of the elongate member, wherein the first conductive wire ends at a distance along the flexible distal portion;
a second conductive wire coupled to the distal tip, wherein the second conductive wire ends distal to the first conductive wire;
wherein the first and second conductive wire are positioned along an outer edge of the flexible distal portion;
whereby, when the flexible distal portion is straightened, the first conductive wire contacts the second conductive wire, thereby enabling energy delivery; and,
whereby, when the flexible distal portion is curved, the first conductive wire does not contact the second conductive wire, thereby disabling energy delivery.
